(19) United States
(12) Patent Application Publication
(10) Pub. No.: US 2006/0293270 A1
(43) Pub. Date: Dec. 28, 2006

(54) Methods and compositions for treating ocular disorders

(76) Inventors: Anthony P. Adamis, Bronxville, NY (US); David R. Guyer, New York, NY (US); Marlene W. Modi, Monmouth Junction, NJ (US)

Correspondence Address: (OSI) EYETECH, INC., 3 TIMES SQUARE 12TH FLOOR, NEW YORK, NY, 10036, US

(21) Appl. No.: 11/472,761

(22) Filed: Jun. 22, 2006

Related U.S. Application Data
(60) Provisional application No. 60/692,727, filed on Jun. 22, 2005.

(51) Int. Cl. A61K 48/00

(52) U.S. Cl. 514/44

(57) ABSTRACT

This invention relates to methods of treating ocular disease. The method of the invention is directed to the administration of an anti-vascular endothelial growth factor (anti-VEGF) compound to treat such disease.

### RELATED APPLICATIONS

This Application claims the benefit of U.S. Provisional Application No. 60/692,727, filed on Jun. 22, 2005 and U.S. Provisional Application. The entire teachings of the above applications are incorporated herein by reference.

### FIELD OF THE INVENTION

This invention relates to compositions and methods of treating ocular disorders, including, but not limited to, age-related macular degeneration (AMD), retinal vein occlusion (RVO), diabetic macular edema (DME), and diabetic retinopathy (DR). The method of the invention is directed to the administration of an anti-vascular endothelial growth factor (anti-VEGF) compound to treat such disorders.

### BACKGROUND OF THE INVENTION

The National Eye Institute and Prevent Blindness America estimated that in 2002, approximately 3.4 million Americans age 40 and older were visually impaired, with over one million being legally blind. See Prevent Blindness America and National Eye Institute, Vision Problems in the U.S. (2002). The prevalence of blindness and vision impairment increases rapidly as people age, particularly in the over-75 age group. According to the National Center for Health Statistics, in 1997, 26% of all nursing home residents in the United States, totaling over 420,000 individuals, had some level of visual impairment. See National Center for Health Statistics, National Nursing Home Survey (1997), available at http://www.cdc.gov/nchs. As a result of demographic changes in the United States, the number of individuals with vision impairment is expected to double in the next three decades. See Prevent Blindness America and National Eye Institute, Vision Problems in the U.S.

Vision impairment causes personal trauma and incapacity, thereby imposing large costs upon society. A study performed by J. M. McNeil in 2001 found that among persons in the United States between the ages of 21 and 64, only 41.5% of persons with visual impairment were employed, as compared to 84% of persons without any disabilities. See U.S. Bureau of the Census, Current Population Reports, P70-61 & P70-73 (2001). The same study found that the average annual earnings of individuals with visual impairment were approximately 31% less than those of persons without any disabilities. See id. In 1998, the National Advisory Eye Council estimated that the economic impact of visual disorders and disabilities in the United States was more than $38.4 billion per year, with $22.3 billion of that amount attributed to direct costs and another $16.1 billion attributed to indirect costs.

Eye disease can be caused by many factors and can affect both the front and back of the eye. In its most extreme cases, eye disease can result either in partial blindness, in which some vision is preserved, or in total blindness. AMD and diabetic retinopathy, including DME, are among the leading causes of significant vision loss. See Prevent Blindness America and National Eye Institute, Vision Problems in the U.S. These diseases deny patients their sight, and, as a result, their ability to live independently and perform daily activities.

AMD is the leading cause of irreversible, severe blindness in patients over the age of 55 in the western world, and affects almost 15 million people in the United States alone. See American Macular Degeneration Foundation, available at http://www.macular.org; Klein et al., Prevalence of Age-related Maculopathy. The Beaver Dam Eye Study, 99 Ophthalmol. 933-43 (1992); Schepens Eye Research Institute, Macular Degeneration Fact Sheet; U.S. Bureau of the Census, 1998 Population Estimates (1998). AMD is caused by the deterioration of the central portion of the retina, known as the macula. There are two types of AMD: dry AMD and wet AMD. While many more people suffer from dry AMD, it accounts for only 10% of the severe vision loss associated with AMD and has no generally accepted treatment. See National Eye Institute, available at http://www.nei.nih.gov. On the other hand, wet AMD is responsible for 90% of the severe vision loss associated with this disease. See id.

There are three subtypes of the wet form of AMD: predominantly classic (affecting approximately 25% of patients suffering from wet AMD), minimally classic (affecting approximately 35% of wet AMD sufferers) and occult (affecting approximately 40% of wet AMD sufferers). See QLT, Inc., available at http://www.qltinc.com/Qltinc/main/mainhome.cfm. Although the specific factors that cause wet AMD are not conclusively known, aging appears to be the most important risk factor. The number of cases of wet AMD will increase significantly as baby boomers age and overall life expectancy increases.

Research of wet AMD shows that vascular endothelial growth factor (“VEGF”) is one of the major factors causing both abnormal blood vessel growth (angiogenesis) and blood vessel leakage in the eye. Specifically, preclinical studies have shown that a) in multiple animal species, including humans, and models, VEGF levels are elevated around growing and leaky blood vessels, b) blocking VEGF results in the prevention and regression of these abnormal vessels in primates and other species and c) VEGF alone is sufficient to trigger the abnormal blood vessel growth that characterizes wet AMD and the blood vessel leakage that characterizes DME. See A. P. Adamis et al., Inhibition of vascular endothelial growth factor prevents retinal ischemia-associated iris neovascularization in a nonhuman primate, 114(1) Arch. Ophthalmol. 66-71 (1996); A. Kvanta et al., Subfoveal fibrovascular membranes in age-related macular degeneration express vascular endothelial growth factor, 37 Invest. Ophthalmol. Vis. Sci. 1929-34 (1996); G. Lutty et al., Localization of vascular endothelial growth factor in human retina and choroids, 114 Arch. Ophthalmol. 971-77 (1996); M. J. Tolentino et al., Intravitreous injections of vascular endothelial growth factor produce retinal ischemia and microangiopathy in an adult primate, 103(11) Ophthalmology 1820-28 (1996); M. J. Tolentino, Vascular endothelial growth factor is sufficient to produce iris neovascularization and neovascular glaucoma in a nonhuman primate, 114(8) Arch. Ophthalmol. 964-70 (1996).

Substantial peer-reviewed research has found high concentrations of VEGF in the eyes of humans afflicted with wet AMD. For example, in a study published by the New England Journal of Medicine, vitreous levels of VEGF were shown to be very high in patients with angiogenic diseases, but were negligible in patients undergoing the same type of surgery for nonangiogenic diseases. See Aiello et al., 331 New. Eng. J. Med. 1480-87 (1994). In a separate study, it was shown that ocular VEGF levels are elevated in patients with active DME. See S. A. Vinores et al., Upregulation of vascular endothelial growth factor in ischemic and non-ischemic human and experimental retinal disease, 12(1) Histol. Histopathol. 99-109 (1997).

Diabetes Mellitus (DM) is an abnormality of blood glucose metabolism due to either reduced insulin production or altered insulin activity. Approximately 15% of the 15 million diabetics in the USA have Type I insulin-dependent diabetes diagnosed before the age of 30. However, the majority of patients are diagnosed after this age with non-insulin dependent diabetes mellitus (NIDDM), or the Type II form. DM results in numerous long-term systemic complications including diabetic retinopathy (DR). DR is broadly classified as either non-proliferative diabetic retinopathy (NPDR), or proliferative diabetic retinopathy (PDR). The differentiation is based on the presence (PDR) or absence (NPDR) of new or abnormal retinal blood vessels. While those with Type I DM experience a very high incidence of severe ocular complications, it is the Type II group who makes up the vast majority of cases with diabetic eye disease simply because of their overall larger numbers (in excess of 12 million in the US alone).

DR is a vascular complication of both types of DM and is correlated with the duration of the underlying endocrine disease. DR remains one of the leading causes of blindness in western societies and vision loss usually results from vitreous hemorrhage, traction retinal detachment or diabetic macular edema (DME). DME can occur with either NPDR or PDR and is the most common cause of diabetic-related visual acuity impairment.

Laser photocoagulation or other surgical modalities can help reduce the risk of moderate (3 or more line) or severe (<20/800) distance visual acuity loss. Panretinal photocoagulation (or scatter laser therapy) is the standard treatment for patients with high risk PDR or patients approaching high risk PDR including some patients with type II diabetes with severe NPDR.

Clinically, DME is defined as retinal thickening within 2 disc diameters of the center of the macula, with or without lipid exudates, and with or without cystoid features. Clinically significant macular edema (CSME) is defined as having one or more of the following features: retinal thickening within 500 μm from the center of the macula; hard exudates within 500 μm of the center of the macula with adjacent retinal thickening; retinal thickening of at least 1 disc area of which at least part is within 1 disc diameter of the center of the macula. Hence, patients with CSME have maculopathy that threatens or affects the center of the macula.

The Early Treatment Diabetic Retinopathy Study (ETDRS) showed that focal laser photocoagulation (direct treatment to microaneurysms, grid treatment to areas on fluorescein angiography of diffuse leakage and areas of non-perfusion judged to be contributing to edema) within thickened retina is beneficial in preventing loss of vision if instituted once CSME develops. Furthermore, the level of visual acuity was not shown to interact with the treatment benefit at the time CSME development. However, it concluded that institution of such therapy prior to development of CSME had no added benefit if one applied treatment once CSME developed.

Despite the benefit of focal laser photocoagulation for CSME, there are approximately 1 out of 6 treated patients who still lose at least 3 lines of visual acuity following this intervention and only approximately 1 out of 8 treated patients gain 3 or more lines of best-corrected distance visual acuity at 3 years after treatment.

Photodynamic therapy (PDT) has received FDA approval for subjects with choroidal neovascularization (CNV) referred to as “predominantly classic” based on the pattern of vascular fluorescence and leakage seen on fluorescein angiogram. Sixty seven percent of the predominantly classic subjects in the PDT group achieved the primary efficacy endpoint—losing less than 15 letters at week 54—compared to 39% of subjects in placebo group (p<0.001). However, this subgroup constitutes only about 25% of all AMD subjects afflicted with subfoveal CNV.

In a dose-finding Phase 1 trial, pegaptanib sodium was given to 15 subjects as single doses ranging from 0.25 mg/eye (6.9 mM) to 3 mg/eye (110 mM), which, due to increasing viscosity with increasing dose, is the maximal dose that can be given in an acceptable volume (0.1 ml) for intravitreous injection with the current formulation. In addition, three repeat doses (4 weeks apart) of 3 mg/eye were given to 21 subjects in two phase 2 studies. There were no local dose-limiting toxicities observed and no systemic toxicity attributed to pegaptanib sodium (sodium pegaptanib injection) in any of the three studies. Approximately 30% of the subjects exhibited a 3, or more, line improvement 3 months after starting treatment.

Two Phase ⅔ randomized, double-masked, controlled, multi-center, comparative trials (EOP1003 and EOP1004) were done to establish the safety and efficacy of intravitreous injections of pegaptanib sodium (0.3, 1 or 3 mg) as compared to sham injection given every 6 weeks.

A total of 1,208 subjects with wet AMD were randomized for enrollment in studies EOP1003 and EOP1004. Data from the first year of these trials demonstrates that pegaptanib sodium was well-tolerated. More than 10,000 intravitreous or sham injections have been administered with 25% of subjects receiving a sham injection. A total of 7,545 intravitreous injections of pegaptanib sodium (0.3, 1 or 3 mg) have been administered during the first year of these two studies. The mean number of injections per subject during the studies ranged from 8.4 to 8.6 of a possible 9 total injections. The median age of subjects participating in EOP1003 and EOP1004 is 77 years.

In December 2004, the FDA approved Macugen® 0.3 mg (pegaptanib sodium injection) for the treatment of neovascular AMD regardless the angiographic subtype. Seventy percent of the subjects in the pegaptanib 0.3 mg group achieved the primary efficacy endpoint—losing less than 15 letters at week 54—compared to 55% of subjects in the usual care group (p<0.001). The control group was considered usual care as PDT was allowed for predominantly classic subjects at investigator's discretion. The mean change in visual acuity was −7.5 letters in the pegaptanib 0.3 mg group and −14.8 letters in the usual care group. No one subgroup of subjects—age, gender, baseline visual acuity, lesion size, lesion subtype, skin or iris pigmentation—drove the overall results observed in the pegaptanib treatment arms. Macugen®, (pegaptanib sodium injection) (Eyetech Pharmaceuticals, NY, N.Y.), a pegylated anti-VEGF aptamer, is described in greater detail in U.S. Pat. Nos. 6,426,335 and 6,051,698, hereby incorporated in their entirety by reference.

In the combined analysis, all pegaptanib sodium doses tested demonstrated statistically significant efficacy compared with control for the clinically relevant primary efficacy endpoint of the proportion of subjects losing less than 15 letters of VA up to 54 weeks. Pegaptanib sodium activity was observed at the 6-week post-injection visit and was sustained throughout the year. There was no evidence to suggest that the overall effect was derived from any one subject subgroup (e.g., baseline visual acuity, lesion subtype, lesion size, or prior treatment with PDT). Mean visual acuity loss at 1 year was reduced in approximately 50% compared to usual care. In the second year, subjects were re-randomized to either continue or discontinue masked therapy for 48 more weeks. The data revealed that the treatment benefit continued throughout the second year of pegaptanib sodium therapy as compared to usual care controls. During the second year, subjects receiving continued Macugen® 0.3 mg were less likely to experience 15 letter loss compared with subjects discontinuing treatment after 1 year.

EOP1002 was an open-label, single-arm, multicenter, exploratory study to investigate the safety and preliminary efficacy of at least 3 consecutive intravitreous injections of pegaptanib sodium (3 mg) given at 6-week intervals in 10 diabetic patients with macular edema (DME). Three to six injections of study drug at 6 week intervals could be given based on the investigator's assessment of clinical need. Investigators could decide not to retreat based on either, lack of apparent efficacy, or good evidence of efficacy (resolution of edema or improvement in vision). Safety and efficacy assessments were to be performed at baseline, at each injection visit, and patients were followed up to week 82.

EOP1005 was a Phase 2 randomized, controlled, double-masked, dose-finding (0.3, 1.0 or 3.0 mg/eye), multi-center, comparative trial, in parallel groups patients with DME involving the center of the macula. Pegaptanib sodium or sham was given to 169 patients (128 treated and 41 sham patients) every 6 weeks by intravitreous injection for 12 to 30 weeks, and patients are still be followed out to 82 weeks after the start of their treatment.

Significant benefits of treatment traditionally judged statistically significant (p<0.05) with 0.3 mg pegaptanib sodium compared with sham treatment between baseline and week 36 were demonstrated for two efficacy endpoints: an increase in mean visual acuity (+4.7 letters versus −0.4 letters; p=0.0419), and a decrease in retinal center point thickness (−68 μm versus +3.7 μm; p=0.0209). In addition, the proportion of patients with an absolute decrease in retinal thickness of ≧75 μm and ≧100 μm in the central part of the central retina were also higher in both the 0.3 mg and 1 mg pegaptanib groups compared with sham (0.3 mg, p=0.0078; 1 mg, p=0.0206). Furthermore, there was a decrease in the need for focal/grid laser therapy at week 12 or later (11 versus 20 patients; p=0.0425).

There is no proven effective therapy for the treatment of DME in patients who have failed to respond to laser therapy. Among the angiogenic growth factors, VEGF is unique in terms of its vascular permeability enhancement, selective endothelial cell mitogenic activity, regulation by hypoxia, advanced glycation end products, insulin-like growth factor, reactive oxygen intermediates, and secretion by most tumor cells. Thus, pegaptanib sodium (anti-VEGF pegylated aptamer) could play a significant role in an ocular disease such as DME by inhibiting vascular leakage.

### SUMMARY OF THE INVENTION

The present invention provides compositions and methods of treating ocular disease, including, but not limited to, macular degeneration, diabetic macular edema, retinal vein occlusion, ischemic retinopathy, diabetic retinal edema, and diabetic retinopathy comprising administering an anti-VEGF agent locally into the eye. In some embodiments, the anti-VEGF agent is an anti-VEGF aptamer and is administered at a dosage of less than 0.3 mg to about 0.003 mg locally into the eye. In some embodiments, the anti-VEGF aptamer is administered at a dosage less than about 0.30 mg. In some embodiments, the anti-VEGF aptamer is administered by intravitreous injection. In some embodiments, the anti-VEGF aptamer is administered every 4-6 weeks, and in other embodiments, the treatment is continued for a period of at least one year. In a particular embodiment, the anti-VEGF aptamer is PEGylated.

According to one embodiment, the present invention provides a method for treating ocular disease comprising administering a therapeutically effective amount of an anti-VEGF agent locally into the eye wherein the treatment is effective to treat occult, minimally classic, and predominantly classic forms of wet macular degeneration, wherein the agent is an aptamer, antibody or antibody fragment.

According to another aspect, the invention provides a pharmaceutical formulation comprising an anti-VEGF aptamer conjugated to a polyethylene glycol in a pharmaceutically acceptable carrier formulation for local administration into the eye, wherein the aptamer is present in the formulation at a concentration of 0.003 mg/90 μl-0.30 mg/90 μl. According to one embodiment, the carrier comprises sodium phosphate and sodium chloride. According to one specific embodiment the carrier comprises 10 mM sodium phosphate and 0.9% sodium chloride.

According to another embodiment, the anti-VEGF agent is administered by intravitreous injection every 4-6 weeks for a period of at least one year and the anti-VEGF agent is an aptamer. The aptamer is conjugated to polyethylene glycol having a molecular weight of about 10-80 Kd or 20-45 Kd.

According to a further embodiment, the anti-VEGF agent is 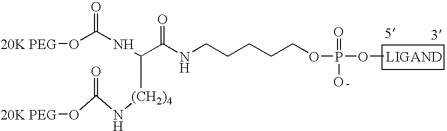
Ligand Component=fCmGmGrArAfUfCmAmGfUmGmAmAfUmGfCfUfUmAfUmAfCmAfUfCfCmG-3′3′-(VEGF ligand)

and the therapeutically effective amount is less than 0.3 mg, 0.003 mg-0.1 mg, or about 0.03 mg or 0.003 mg.

According to another embodiment, the dose is effective to achieve a vitreous concentration of the anti-VEGF aptamer of about 10-30 ng/mL.

According to another embodiment, the dose is effective to maintain a vitreous concentration of the anti-VEGF aptamer of about 10-30 ng/mL throughout a 6 week dosing interval.

Definitions

By “phototherapy” is meant any process or procedure in which a patient is exposed to a specific dose of light of a particular wavelength, including laser light, in order to treat a disease or other medical condition.

By “photodynamic therapy” or “PDT” is meant any form of phototherapy that uses a light-activated drug or compound, referred to herein as a photosensitizer, to treat a disease or other medical condition characterized by rapidly growing tissue, including the formation of abnormal blood vessels (i.e., angiogenesis). Typically, PDT is a two-step process that involves local or systemic administration of the photosensitizer to a patient followed by activation of the photosensitizer by irradiation with a specific dose of light of a particular wavelength. Photodynmaic therapies and photosensitizers are known in the art, as disclosed, for example, in U.S. Pat. Nos. 5,756,541, 5,798,349, 6,599,891, and 6,610,670 and PCT Publications WO 00/00204, WO 00/73308, WO 01/74818, WO 02/096366, WO 02/096417, WO 03/028629, WO 03/028628, WO 02/062386, WO 03/045432, and WO 01/58240, which are hereby incorporated in their entirety by reference.

By “anti-VEGF agent” is meant a compound that inhibits the activity or production of vascular endothelial growth factor (“VEGF”).

By “photosensitizes” or “photoactive agent” is meant a light-absorbing drug or other compound that upon exposure to light of a particular wavelength becomes activated thereby promoting a desired physiological event, e.g., the impairment or destruction of unwanted cells or tissue.

By “thermal laser photocoagulation” is meant a form of photo-therapy in which laser light rays are directed into the eye of a patient in order to cauterize abnormal blood vessels in the eye to seal them from further leakage.

By “effective amount” is meant an amount sufficient to treat a symptom of an ocular disease.

The term “light” as used herein includes all wavelengths of electromagnetic radiation, including visible light. Preferably, the radiation wavelength is selected to match the wavelength(s) that excite(s) the photosensitizer. Even more preferably, the radiation wavelength matches the excitation wavelength of the photosensitizer and had low absorption by non-target tissues.

### DETAILED DESCRIPTION OF THE INVENTION

According to the present invention, the ID₅₀ and IC₅₀ for pegaptanib sodium in two established murine models of ocular angiogenesis: the murine model of corneal neovascularization and the murine model of retinopathy of prematurity has been established. Following twice daily IP injections of pegaptanib sodium, corneal neovascularization was inhibited at an ID₅₀ of 22.50 mg/kg and an IC₅₀ of 0.50 nM (corneal tissue concentration of 5.48 ng/mL). In the murine model of retinal neovascularization, retinal neovascularization was inhibited with an ID₅₀ of 3.70 mg/kg and an IC₅₀ of 0.21 nM (ocular concentration of 1.95 ng/mL). Such determinations had previously been outside the range of quantitation.

According to the invention, therapeutically effective compositions and methods for treating ocular disorders are provided wherein an anti-VEGF agent is administered locally at a dose of less than 0.3 mg. In one embodiment the anti-VEGF agent is pegaptanib sodium and is administered in the range of 0.003 mg to less than about 0.3 mg.

According to one embodiment, the dose is effective to achieve vitreous concentrations of pegaptanib sodium within about 10 to 30 ng/mL during a 6 week dosing interval. According to another embodiment, the dose is effective to maintain vitreous concentrations of pegaptanib sodium within about 10 to 30 ng/mL throughout the entire 6 week dosing interval.

A variety of anti-VEGF therapies that inhibit the activity or production of VEGF, including aptamers and VEGF antibodies, are available and can be used in the methods of the present invention. The preferred anti-VEGF agents are nucleic acid ligands of VEGF, such as those described in U.S. Pat. Nos. 6,168,778 B1; 6,147,204; 6,051,698; 6,011,020; 5,958,691; 5, 817,785; 5,811,533; 5,696,249; 5,683,867; 5,670,637; and 5,475,096, hereby incorporated in their entirety by reference. A particularly preferred anti-VEGF agent is Macugen® (pegaptanib sodium injection) (EYE001, previously referred to as NX1838), which is a modified, pegylated aptamer that binds with high affinity to the major soluble human VEGF isoform and has the general structure shown in FIG. 1 (described in U.S. Pat. No. 6,168,788; Journal of Biological Chemistry, Vol. 273(32): 20556-20567 (1998); and In Vitro Cell Dev. Biol. Animal Vol. 35:533-542 (1999)).

Alternatively, the anti-VEGF agents may be, for example, VEGF antibodies or antibody fragments, such as those described in U.S. Pat. Nos. 6,100,071; 5,730,977; and WO 98/45331. Other suitable anti-VEGF agents or compounds that may be used in combination with anti-VEGF agents according to the present invention include, but are not limited to, antibodies specific to VEGF receptors (e.g., U.S. Pat. Nos. 5,955,311; 5,874,542; and 5,840,301); compounds that inhibit, regulate, and/or modulate tyrosine kinase signal transduction (e.g., U.S. Pat. No. 6,313,138 B1); VEGF polypeptides (e.g., U.S. Pat. No. 6,270,933 B1 and WO 99/47677); oligonucleotides that inhibit VEGF so expression at the nucleic acid level, for example antisense RNAs (e.g., U.S. Pat. Nos. 5,710,136; 5, 661,135; 5,641,756; 5,639,872; and 5,639,736); retinoids (e.g., U.S. Pat. No. 6,001,885); growth factor-containing compositions (e.g., U.S. Pat. No. 5,919,459); antibodies that bind to collagens (e.g., WO 00/40597); and various organic compounds and other agents with angiogenesis inhibiting activity (U.S. Pat. Nos. 6,297,238 B1; 6,258, 812 B1; and 6,114,320).

The anti-VEGF agents can also be administered topically, for example, by patch or by direct application to the eye, or by iontophoresis. The anti-VEGF agents may be provided in sustained release compositions, such as those described in, for example, U.S. Pat. Nos. 5,672,659 and 5,595,760. The use of immediate or sustained release compositions depends on the nature of the condition being treated. If the condition consists of an acute or over-acute disorder, treatment with an immediate release form will be preferred over a prolonged release composition. Alternatively, for certain preventative or long-term treatments, a sustained released composition may be appropriate.

The anti-VEGF agent may also be delivered using an intraocular implant. Such implants may be biodegradable and/or biocompatible implants, or may be non biodegradable implants. The implants may be permeable or impermeable to the active agent, and may be inserted into a chamber of the eye, such as the anterior or posterior chambers or may be implanted in the sclera, transchoroidal space, or an avascularized region exterior to the vitreous. In a preferred embodiment, the implant may be positioned over an avascular region, such as on the sclera, so as to allow for transcleral diffusion of the drug to the desired site of treatment, e.g. the intraocular space and macula of the eye. Furthermore, the site of transcleral diffusion is preferably in proximity to the macula.

Examples of implants for delivery of an anti-VEGF agent include, but are not limited to, the devices described in U.S. Pat. Nos. 3,416,530; 3,828,777; 4,014,335; 4,300,557; 4,327,725; 4,853,224; 4,946,450; 4,997,652; 5,147,647; 5,164,188; 5,178,635; 5,300,114; 5,322,691; 5,403,901; 5,443,505; 5,466,466; 5,476,511; 5,516,522; 5,632,984; 5,679,666; 5,710,165; 5,725,493; 5,743, 274; 5,766,242; 5,766,619; 5,770,592; 5773,019; 5,824,072; 5,824,073; 5,830,173; 5,836,935; 5,869,079, 5,902,598; 5,904,144; 5,916,584; 6,001,386; 6,074,661; 6,110,485; 6,126,687; 6,146,366; 6,251,090; and 6,299,895, and in WO 01/30323 and WO 01/28474, all of which are incorporated herein by reference.

When administered directly to the eye, the dosage range is less than 0.3 mg, such as about 0.003 mg to about 0.3 mg per eye. The dosage may be administered as a single dose or divided into multiple doses. In general, the desired dosage should be administered at set intervals for a prolonged period, usually at least over several weeks, although longer periods of administration of several months or more may be needed.

According to another embodiment, the present invention features a method for treating a patient suffering from an ocular disease, which method includes the following steps: (a) administering to the patient an effective amount of an anti-VEGF aptamer; and (b) providing the patient with phototherapy, such as photodynamic therapy or thermal laser photocoagulation as further described in PCT WO 03/039404, incorporated in its entirety by reference.

In one embodiment of the invention, the photodynamic therapy (PDT) includes the steps of: (i) delivering a photosensitizer to the eye tissue of a patient; and (ii) exposing the photosensitizer to light having a wavelength absorbed by the photosensitizer for a time and at an intensity sufficient to inhibit neovascularization in the patient's eye tissue. A variety of photosensitizers may be used, including but not limited to, benzoporphyrin derivatives (BPD), monoaspartyl chlorine, zinc phthalocyanine, tin etiopurpurin, tetrahydroxy tetraphenylporphyrin, and porfimer sodium (PHOTOFRIN), and green porphyrins.

In a related aspect, the present invention provides a method for treating an ocular disease in a patient, which method involves administering to the patient: (a) an effective amount of an anti-VEGF aptamer; and (b) a second compound capable of diminishing or preventing the development of unwanted neovasculature. The anti-VEGF agents or other compounds that may be combined with anti-VEGF aptamers include, but are not limited to: antibodies or antibody fragments specific to VEGF; antibodies specific to VEGF receptors; compounds that inhibit, regulate, and/or modulate tyrosine kinase signal transduction; VEGF polypeptides; oligonucleotides that inhibit VEGF expression at the nucleic acid level, for example antisense RNAs; retinoids; growth factor-containing compositions; antibodies that bind to collagens; and various organic compounds and other agents with angiogenesis inhibiting activity. According to one embodiment, the second agent comprises an anti-PDGF aptamer as described further in PCT WO 2005/020972, hereby incorporated in its entirety by reference.

The features and other details of the invention will now be more particularly described and pointed out in the following examples describing preferred techniques and experimental results. These examples are provided for the purpose of illustrating the invention and should not be construed as limiting.

### EXAMPLES

Macugen® ((OSI) Eyetech, N.Y., N.Y.) is formulated at 0.3 mg/90 μl, 0.03 mg/90 μl or 0.003 mg/901 μl and presented in USP Type I glass barrel syringes sealed with a bromobutyl rubber plunger stopper. The syringe has a fixed 27-gauge needle with a rubber needle shield (tip cap) and a rigid plastic outer shield. The stoppered syringe is packaged in a foil pouch. A plastic plunger rod and flange adapter are also supplied for administration purposes. These components are provided in a separate foil pouch. Use of the flange is optional and is not required to administer the injection. The drug product is preservative-free and intended for single use by intravitreous injection only. The product should not be used if cloudy or if particles are present.

Active Ingredient: Pegaptanib Sodium Injection formulated as:
0.0347 mg/mL solution to deliver a dose of 0.003 mg pegaptanib sodium injection
0.347 mg/mL solution to deliver a dose of 0.03 mg pegaptanib sodium injection
3.47 mg/mL solution to deliver a dose of 0.3 mg pegaptanib sodium injection

Excipients: Sodium Chloride, USP
Sodium Phosphate Monobasic, Monohydrate, USP
Sodium Phosphate Dibasic, Heptahydrate, USP
Sodium Hydroxide, USP (as needed)
Hydrochloric acid, USP (as needed)
Water for injection, USP
   Preparation
   The drug product pegaptanib sodium is a ready-to-use sterile solution provided in a single-use glass syringe. Administration of the syringe contents involves attaching the threaded plastic plunger rod to the rubber stopper inside the barrel of the syringe. The rubber end cap is then removed to allow administration of the product. An optional flange is provided for administrative purposes.
   Treatment Regimen and Duration
   Pegaptanib sodium will be administered as 90 μl (nominal delivered volume) intravitreous injections every 6 weeks.
   Intravitreous Injection

1% Mydriacyl and 2.5% Phenylephrine are applied topically to the study eye to achieve adequate pupillary dilation. Two to three drops of 50% saline diluted 10% povidone-iodine (betadine) solution are instilled into the eye. In the event of allergy to iodine, a drop of topical antibiotic is placed on the conjunctiva in place of iodine. A subconjunctival injection of 0.5 ml 2% xylocalne without epinephrine is administered in the inferotemporal quadrant in all patients −3.0 to 3.5 mm from the limbus in aphakic/pseudophakic patients, and 3.5 to 4.0 mm in phakic patients. Investigators are instructed to select one of two pre-injection procedures (Options A and B, below). For patients with iodine allergy, investigators are required follow Option A, instilling one additional drop of antibiotic instead of povidone-iodine.
A. Administer topical ofloxacin, levofloxacin, or an antibiotic drop with comparable antimicrobial coverage for three days prior to the treatment followed by three consecutive drops of antibiotic and several drops of 5% povidone-iodine immediately before the treatment
B. Administer three consecutive drops of antibiotic and a 5% povidone-iodine flush of the fomices and caruncle with at least 10 cc of solution just prior to treatment.

Prior to treatment, topical antibiotic drops are administered 3 times separated by at least 5 minutes within one hour prior to treatment.

For patients who are prepared under Option A, following the last dose of antibiotic, the investigator instills two or three drops of 5% povidone-iodine into the eye. Using sterile gloves and cotton-tip applicators soaked in 5% povidone iodine, the investigator scrubs the eyelids, the upper and lower eyelid margins, and the caruncle 3 times. In the event of allergy to iodine, one additional drop of antibiotic is instilled instead of povidone-iodine.

For patients who are prepared under Option B, the investigator waits at least 5 minutes after the last dose of antibiotic to perform a 5% povidone-iodine flush, irrigating the fornices and the caruncle with at least 10 cc of 5% povidone-iodine using a forced stream from a syringe connected to an angio-catheter to effect mechanical debridement.

After changing gloves, the investigator isolates the ocular field with a drape, pinning the eyelashes to the eyelids, and places one or two drops of 5% povidone-iodine on the ocular surface at the intended treatment site. An eyelid speculum is used for all injections.

Treatment Administration—Pegaptanib Sodium

Active Drug: Following the administration of subconjunctival xylocalne, the rubber stopper covering the needle is removed and the entire volume of the drug is injected. The needle of the pegaptanib sodium syringe is inserted until the tip is just visualized through the dilated pupil.

We claim:

1. A method for treating an ocular disease in a patient comprising:
(a) Administering less than 0.3 mg of the anti-VEGF aptamer identified by the following structure 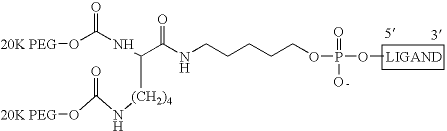
Ligand Component=fCmGmGrArAfUfCmAmGfUmGmAmAfUmGfCfUfUmAfUmAfCmAfUfCfCmG-3′3′-(VEGF ligand)

2. The method of claim 1 wherein the ocular disease is selected from the group consisting of macular degeneration, diabetic macular edema, retinal vein occlusion, ischemic retinopathy, diabetic retinal edema, and diabetic retinopathy.

3. The method of claim 1 wherein the ocular disease is macular degeneration.

4. The method of claim 1 wherein the aptamer is administered via intravitreous injection.

5. The method of claim 1 wherein the aptamer is administered at a dosage of about 0.1 mg to about 0.003 mg.

6. The method of claim 1 wherein the aptamer is administered every 4-6 weeks.

7. The method of claim 1 wherein the aptamer comprises pegaptanib sodium in a pharmaceutically acceptable carrier formulation for local administration into the eye, wherein the aptamer is present in the formulation at a concentration of 0.003 mg/90 μl-0.30 mg/90 μl.

8. The method according to claim 6 wherein the carrier comprises sodium phosphate and sodium chloride.

9. The method of claim 7 wherein the carrier comprises 10 mM sodium phosphate and 0.9% sodium chloride.

10. The method of claim 1 wherein the dose is effective to achieve a vitreous concentration of the aptamer of about 10-30 ng/mL.
